(19) United States
(12) Patent Application Publication
(10) Pub. No.: US 2026/0272954 A1
(43) Pub. Date: Sep. 17, 2026

(54) ANALGESIC COMPOUNDS COMPRISING CHARGED PARTICLES AND METHOD OF MANUFACTURE THEREOF

(71) Applicant: UNIVERSITY OF CONNECTICUT, Farmington, CT (US)

(72) Inventors: Lakshmi S. NAIR, New Britain, CT (US); Joseph WALKER, Avon, CT (US); Reinier A. GONZALEZ HEREDIA, Meriden, CT (US); Mu-Ping NIEH, Mansfield, CT (US); Devika KISHNAN, New Britain, CT (US)

(21) Appl. No.: 19/474,818

(22) PCT Filed: Apr. 15, 2024
(86) PCT No.: PCT/US2024/024657

§ 371 Date: Oct. 13, 2025

Related U.S. Application Data
(60) Provisional application No. 63/459,057, filed on Apr. 13, 2023.

(51) Int. Cl. A61K 31/00; A61K 47/24

(52) U.S. Cl. CPC A61K 31/658; A61K 47/24

(57) ABSTRACT

Disclosed herein is an analgesic composition comprising a nanoparticle and/or a microparticle that comprises a charged lipid and/or a supporting lipid; where the charged lipid comprises an electrically charged group. Disclosed herein too is a method of reducing joint inflammation, swelling and/or pain in a subject, the method comprising administering a therapeutically effective amount an analgesic composition to the subject; where the analgesic composition comprises a nanoparticle and/or a microparticle that comprises a charged lipid and/or a supporting lipid; where the charged lipid comprises an electrically charged group.

### CROSS-REFERENCE TO RELATED APPLICATIONS

This disclosure claims priority to U.S. Provisional Application No. 63/459,057, filed on Apr. 13, 2024, the entire contents of which are incorporated herein in their entirety.

### GOVERNMENT SUPPORT

The invention was made with government support under grant numbers R01-AR075143 awarded by the National Institutes of Health. The government has certain rights in the invention.

### BACKGROUND

This disclosure relates to analgesic compounds comprising charged particles and to methods of manufacture thereof. In particular, this disclosure relates to compounds, compositions, and methods for modulating pain, and their use in the prevention and/or treatment of pain and pain related illnesses.

Medications such as pain relievers and anti-inflammatory drugs may be prescribed to control pain and inflammation. Oral non-steroidal, anti-inflammatory drugs include medications classified as non-steroidal anti-inflammatory drugs (NSAIDs) such as ibuprofen, naproxen, and aspirin, which are commonly used to alleviate pain and reduce inflammation. However, these have serious side effects such as gastrointestinal problems. Opioids, while potent pain relievers, are a separate class of medications and are not classified as NSAIDs. However, these opioids can have serious side effects, such as the risk of addiction, respiratory depression, and constipation. Local anesthetics (LA) are potential molecules for managing pain; this is a non-addictive option, but these anesthetics are short acting. They also exhibit side effects such as local inflammation.

Osteoarthritis is a degenerative joint disorder characterized by a gradual breakdown of cartilage, the tissue that cushions the ends of bones within a joint. As the cartilage wears away, bones begin to rub against each other, leading to pain, stiffness, and reduced joint flexibility. This condition commonly affects weight-bearing joints such as the knees, hips, and spine, but it can also impact other joints over time. The primary risk factors for osteoarthritis include aging, genetics, joint injuries, and obesity. While there is no cure for osteoarthritis, various treatment options aim to manage symptoms and improve joint function. Non-pharmacological approaches include exercise, physical therapy, and weight management to alleviate stress on joints.

In severe cases, surgical interventions like joint replacement may be considered. Overall, a comprehensive approach involving lifestyle modifications, therapeutic interventions, and medical management is often employed to enhance the quality of life for individuals dealing with osteoarthritis.

There is a therefore a need for improved compositions that offer simplified routes for better pain management.

### SUMMARY

Disclosed herein is an analgesic composition comprising a nanoparticle and/or a microparticle that comprises a charged lipid and/or a supporting lipid; where the charged lipid comprises an electrically charged group.

Disclosed herein too is a method of manufacturing particles, the method comprising dissolving a lipid in a first solvent; where the lipid is a charged lipid, a supporting lipid, or a combination of the charged lipid and the supporting lipid; evaporating the first solvent to leave behind a powder or film; adding to the powder or film a second solvent; heating the second solvent containing the powder and the film to a temperature greater than about 100° C. or to a temperature higher than the melting point of the lipid; and agitating the aqueous solution with a rotary agitator at about 1,000 to about 15,000 revolutions per minute to form the particles; where the particles are nanoparticles and/or microparticles.

Disclosed herein too is a method of reducing joint inflammation, swelling and/or pain in a subject, the method comprising administering a therapeutically effective amount an analgesic composition to the subject; where the analgesic composition comprises a nanoparticle and/or a microparticle that comprises a charged lipid and/or a supporting lipid; where the charged lipid comprises an electrically charged group.

### BRIEF DESCRIPTION OF THE FIGURES

FIG. 1A shows properties of DPPC nanoparticles. The transmission electron microscopy (TEM) image indicates vesicular structures of different sizes taken at magnifications of 3000×, 6000× and 12,000×;

FIG. 1B is a small angle xray scattering spectrum that shows the synchronous peaks indicating the formation of bilayers and stacking of bilayers to form multilamellar vesicles (MLVs);

FIG. 2A shows the properties of charged particles, DPPC:PA at a mass ratio of DPPC:PA=1:1. The DPPC:PA nanoparticles shows vesicular morphology;

FIG. 2B is a small angle x-ray scattering spectrum that shows the multilamellar structure has been effectively decoupled in the presence of PA;

FIG. 3A shows the properties of charged DPPC-PEA-PA NPs at a mass ratio of DPPC:PEA:PA=1:1:0.5. The TEM image indicates nanoparticles with vesicular morphology;

FIG. 3B is a small angle X-ray scattering spectrum, which indicates that the addition of PEA would re-induce the stacking of the bilayers (forming MLVs);

FIG. 4A shows the TEM micrograph of DPPC:PA nanoparticles before complexation; FIG. 4B is a micrograph depicts the DPPC:PA nanoparticles after complexation with QX-314;

FIG. 5 shows the percentage of QX-314 in the supernatant after 1 hour of incubation in QX-314 solution; and

FIG. 6 is a graph that shows the percentage of QX-314 released in the water as a function of time from DPPC-PA nanoparticles and from QX-314 solution.

### DETAILED DESCRIPTION

### Definitions

A charged lipid is a type of lipid molecule that contains an electrically charged group. Lipids are a diverse group of organic compounds that are insoluble in water but soluble in organic solvents. They play valuable roles in biological systems, including energy storage, cell membrane structure, and signaling. Charged lipids have a polar head group that carries a positive or negative charge. These head groups can interact with water molecules and other charged molecules, influencing the properties and functions of the lipid. Lipids are characterized by their hydrophobic (water-repelling) nature, which is due to their nonpolar hydrocarbon chains.

The term “endocannabinoid” encompasses at least one endocannabinoid, an endocannabinoid analogue, a pharmaceutically acceptable salt of an endocannabinoid, or a combination thereof. Endocannabinoids are naturally occurring cannabinoids produced by the body. The endocannabinoids primarily target cannabinoid receptors are located on the cell surface. The term endocannabinoid encompasses one or more endocannabinoids, one or more analogues of the endocannabinoid, one or more pharmaceutically acceptable salts of the endocannabinoid, or a combination thereof.

The term “cannabinoid” encompasses a group of chemical compounds that interact with the endocannabinoid system in the human body. They can be classified into three main types a) endocannabinoids; b) phytocannabinoids; and c) synthetic cannabinoids. Cannabinoids exert their effects by interacting with cannabinoid receptors, primarily CB1 and CB2 receptors, which are part of the endocannabinoid system. CB1 receptors are mainly found in the central nervous system, while CB2 receptors are more prevalent in peripheral tissues, particularly immune cells. The term cannabinoid encompasses one or more cannabinoids, one or more analogues of the cannabinoid, one or more pharmaceutically acceptable salts of the cannabinoid, or a combination thereof.

The term “local anesthetic” includes a local anesthetic, an analogue of the local anesthetic, a pharmaceutically acceptable salt of the local anesthetic, or a combination thereof.

An analogue of a chemical compound is essentially a related compound that shares some structural or functional similarities with the original compound.

The term “vesicular morphology” refers to a specific structural appearance characterized by the formation of vesicles. Vesicles are small, membrane-bound sacs that can be formed by various substances, including lipids and proteins, in biological and synthetic systems.

Nanoparticles as described herein include particles having a particle size of about 0.5 to about 100 nanometers. The diameter of the particles is generally considered to be a measure of particle size. Particle size may be determined by small angle xray scattering, scanning electron microscopy and/or transmission electron microscopy.

Microparticles as described herein include particles having a particle size of about 100.1 to about 100,000 nanometers. The diameter of the particles is generally considered to be a measure of particle size. Particle size may be determined by small angle xray scattering, scanning electron microscopy and/or transmission electron microscopy. The term “particles” as used herein encompasses both nanoparticles and microparticles. The term “particle” is used to describe phase separated endocannabinoids, cannabinoids and/or local anesthetics that are dispersed in a solution for administration into the body of a living being. The solution of the particles may be considered to be a suspension.

The term “therapeutically effective” is used to describe the efficacy of a pharmaceutical or therapeutic intervention. It refers to the ability of a particular composition or method to produce a beneficial or desired therapeutic effect in the treatment, prevention, or alleviation of a specific medical condition or disease.

Disclosed herein is an anesthetic and/or analgesic composition (hereinafter analgesic composition) that comprises nanoparticles and/or microparticles that comprise a charged lipid that is complexed with a local anesthetic. In an embodiment, the anesthetic and/or analgesic composition may comprise nanoparticles and/or microparticles that may contain a supporting lipid in addition to the charged lipid. The nanoparticles and/or microparticles may include a cannabinoid and/or an endocannabinoid and/or a local anesthetic. The local anesthetic is optional. The nanoparticles and/or microparticles may be suspended in a fluid and can be delivered periarticularly or intraarticularly to a desired location in a living being.

The nanoparticles have a vesicular structure. In an embodiment, the nanoparticles have a bilayer vesicular structure. In yet another embodiment, the nanoparticles comprise multilamellar vesicles, where the multilamellar vesicles comprise stacked bilayers. Stacked bilayers in a vesicular structure refer to layers of lipid bilayers that are arranged in a stacked or layered configuration within a vesicle. Multilamellar vesicles are vesicular structures that contain multiple concentric layers of lipid bilayers. Each layer comprises phospholipids arranged with their hydrophobic tails facing inward and their hydrophilic heads facing outward, similar to cell membranes.

Local anesthetics are charged molecules and can form complexes with charged lipids. This ability to undergo complex interactions with charged lipids may be advantageously used to produce nanoparticles and/or microparticles in a fluid such as a solvent. The solvent is preferably water or an aqueous solvent. The term “local anesthetic” includes the compound, salts or derivatives thereof.

In an embodiment, the endocannabinoids and/or cannabinoids can form separate nanoparticles from those nanoparticles that contain charged lipids and/or supporting lipids. In another embodiment, the endocannabinoid, the cannabinoid and/or local anesthetic molecules can form complexes with the charged lipids and/or supporting lipids in a nanoparticle. In yet another embodiment, the analgesic composition can comprise first nanoparticles that contain endocannabinoids and/or cannabinoids without the lipids (charged and/or supporting lipids), second nanoparticles that contain endocannabinoids and/or cannabinoids with the lipids (charged and/or supporting lipids) and/or third nanoparticles that contain only lipids (the charged and/or supporting lipids).

Charged lipids are a category of lipids that contain charged functional groups, such as phosphate or sulfate groups, in addition to their lipid components. The charged lipids have naked positive or negative charge to complex negative or positive molecules respectively.

The lipid components may comprise fatty acids (fatty acids are long hydrocarbon chains with a carboxylic acid group at one end. They can be saturated (no double bonds) or unsaturated (contain one or more double bonds)), triglycerides, steroids, lipoproteins, phospholipids, glycolipids, waxes (e.g., esters of long-chain fatty acids and long-chain alcohols), or a combination thereof. The term charged lipid encompasses one or more charged lipid, one or more analogues of the charged lipid, one or more pharmaceutically acceptable salts of the charged lipid, or a combination thereof.

The charged lipids include phospholipids (these have a phosphate group in their head region, which can be further modified with various functional groups), sphingolipids (these are considered charged lipids due to their polar head group), glycolipids (these are lipids that contain a carbohydrate moiety along with a lipid component), or a combination thereof. Examples of phospholipids include phosphatidylcholine (PC), phosphatidylserine (PS), phosphatidylethanolamine (PE), phosphatidylinositol (PI), or a combination thereof.

Examples of sphingolipids include sphingomyelin (e.g., contains a phosphorylcholine head group), ceramides (e.g., these have a sphingosine backbone with various head groups, including phosphate or sugar groups), or a combination thereof.

Examples of glycolipids include gangliosides (e.g., complex glycolipids with sialic acid residues), cerebrosides (e.g., simple glycolipids composed of a ceramide and a single sugar residue), or a combination thereof.

An example of the charged lipid is L-α-phosphatidic acid [PA]; oleoyl-L-α-lysophosphatidic acid; 1,2-diarachidonoyl-sn-glycero-3-[phosphor-rac-(1-glycerol)] (20:4 PG); tetradecanoic acid, 1,1′,1″,1″-{(2R,14R)-5,8,11,-trihydroxy-5,11-dioxido-4,6,10,12-tetraoxa-5,11-diphosphapentadecane-1,2,14,15-tetrayl] ester (Tetramyristoylcardiolipin); (R)-2,3-bis(((9Z,12Z)-octadeca-9,12-dienoyl)oxy)propyl ((1S,2R,3R,4S,5S,6R)-2,3,4,5,6-pentahydroxycyclohexyl) phosphate (Phosphatidylinositol); (1,2-dioleoyl-3-trimethylammonium-propane (POPS); N-(2-hydroxyethyl)-N,N-dimethyl-2,3-bis(oleoyloxy) propan-1-aminium bromide (DORI); O,O′-ditetradecanoyl-N-(α-trimethylammonioacetyl) diethanolamine chloride (DC-6-14); 1,2-di-O-octadecenyl-3-trimethylammonium propane (DOTMA); N1-[2-((1S)-1-[(3-aminopropyl)amino-4-[di(3-amino-propyl)amino]butylcarboxamido)ethyl]-3,4-di[oleyloxy]-benzamide (MVL5), or a combination thereof.

The charged lipid may be present in the analgesic composition in an amount of about 0.1 to about 100 weight percent (wt %), about 0.5 to about 97 wt %, about 0.2 to about 85 wt %, about 1 to about 80 wt %, about 2 to about 70 wt %, about 3 to about 60 wt %, about 3.5 to about 55 wt %, about 4 to about 50 wt %, about 4.5 to about 45 wt %, about 5 to about 40 wt %, about 5.5 to about 35 wt %, about 6 to about 30 wt %, about 6.5 to about 25 wt %, about 7 to about 20 wt %, about 7.5 to about 18 wt %, about 8 to about 15 wt %, and about 9 to about 14 wt %, based on a total weight of the analgesic composition.

In a preferred embodiment, the charged lipid is used in the analgesic composition in an amount of 10 to 100 weight percent, preferably 15 to 60 wt %, and more preferably 17 to 30 wt %, based on the total weight of the composition. The analgesic composition includes the charged lipid, the supporting lipid, the endocannabinoid and/or the cannabinoid. The total weight of the analgesic composition is therefore the sum of the weights of the charged lipid, the supporting lipid, the endocannabinoid and/or endocannabinoid.

A supporting lipid, also known as a structural lipid, is a type of lipid molecule that plays a crucial role in maintaining the structural integrity and functionality of biological membranes. The supporting lipid may or may not have charge. The supporting lipid allow us to control the amount of charge on the surface to modulate the extent of complexation. It also plays a role in nanoparticle structure, composition and integrity. Biological membranes, such as cell membranes, are composed of a lipid bilayer that separates the interior of the cell or organelle from its external environment. Supporting lipids include various types of phospholipids, glycolipids, and cholesterol. These lipids are useful for creating a stable membrane structure and for regulating membrane fluidity and permeability. Examples of supporting lipids include phospholipids (these have a hydrophilic (water-attracting) head group and two hydrophobic (water-repellent) fatty acid tails. The arrangement of phospholipids in a lipid bilayer provides a barrier that separates the inside and outside of cells, creating a selective permeability barrier), glycolipids (these lipids contain a carbohydrate group along with a lipid component), cholesterol, or a combination thereof. The term supporting lipid encompasses one or more supporting lipid, one or more analogues of the supporting lipid, one or more pharmaceutically acceptable salts of the supporting lipid, or a combination thereof.

An example of a supporting lipid that may be used in the analgesic composition includes lipid molecule 1,2-dipalmitoyl-sn-glycero-3-phosphocholine [DPPC], 1,2-dimyristoyl-sn-glycero-3-phosphocholine [DMPC], 1,2-distearoyl-sn-glycero-3-phosphocholine [DSPC], 1,2-dioleoyl-sn-glycero-3-phosphocholine [DOPC], or a combination thereof.

The supporting lipid may be present in the analgesic composition in an amount of about 0.1 to about 100 weight percent (wt %), about 0.5 to about 97 wt %, about 0.2 to about 85 wt %, about 1 to about 80 wt %, about 2 to about 70 wt %, about 3 to about 60 wt %, about 3.5 to about 55 wt %, about 4 to about 50 wt %, about 4.5 to about 45 wt %, about 5 to about 40 wt %, about 5.5 to about 35 wt %, about 6 to about 30 wt %, about 6.5 to about 25 wt %, about 7 to about 20 wt %, about 7.5 to about 18 wt %, about 8 to about 15 wt %, and about 9 to about 14 wt %, based on a total weight of the analgesic composition.

In a preferred embodiment, the supporting lipid may be present in the nanoparticles in an amount of 10 to 100 weight percent, preferably 30 to 90 wt %, preferably 32 to 80 wt %, and more preferably 35 to 45 wt %, based on the total weight of the composition.

In an embodiment, the composition may contain nanoparticles and/or microparticles that contain endocannabinoids, cannabinoids, or a combination thereof.

Endocannabinoids are naturally occurring compounds within the bodies of living beings that interact with the endocannabinoid system (ECS). The ECS is a complex cell-signaling system that plays a crucial role in regulating a range of physiological processes to maintain homeostasis, or balance, in the body. Endocannabinoid ligands include endogenously produced lipids that activate two distinct “direct” endocannabinoid receptors, CB1 and CB2. CB1 receptors are primarily found in the central nervous system, including the brain. It is to be noted that endocannabinoid like molecules may not have to activate CB1 and CB2, they may function differently to provide analgesic and anti-inflammatory properties. The activation of CB1 receptors can modulate the perception of pain and affect the transmission of pain signals.

CB2 receptors are mainly located in peripheral tissues, particularly in immune cells. CB2 receptor activation is associated with anti-inflammatory effects, which can contribute to pain relief in conditions involving inflammation.

Endocannabinoids and endocannabinoid like molecules that interact with the ECS system to provide anti-inflammatory, analgesic (pain-relieving) and/or swelling relief include N-acylethanolamines (NAEs), and N-acyl amino acids their analogues. N-acylethanolamines are fatty acid amides that are naturally produced in the body in response to various stimuli, including inflammation. Examples of N-acylethanolamines and its analogues that can be used for their anti-inflammatory effect include anandamide (AEA) 2-arachidonoylglycerol (2-AG), 2-oleoyl glycerol (2-OG), palmitoylethanolamide (PEA), oleoylethanolamide (OEA), linoleoylethanolamide (LEA), adelmidrol (N,N′-Bis(2-hydroxyethyl) nonanediamide), N-stearoyl ethanolamine (SEA), N-arachidonoyl glycine (NArGly), N-arachidonoylserine (NArS), N-acyl taurine, N-acyl dopamine, N-acyl alanine, or a combination thereof.

In an exemplary embodiment, the preferred endocannabinoid used in the analgesic composition is N-palmitoylethanolamide. A main target of N-palmitoylethanolamide is the peroxisome proliferator-activated receptor alpha (PPAR-α). N-palmitoylethanolamide also has affinity to cannabinoid-like G-coupled receptors GPR55 and GPR119. Palmitoylethanolamide (or other structurally related N-acylethanolamines) enhances anandamide activity by an “entourage effect”.

Cannabinoids are chemical compounds found in the cannabis plant. Some cannabinoids may also be used for their anti-inflammatory effect. Examples of such cannabinoids or cannabinoid analogues include cannabigerol (CBG), cannabichromene (CBC), trans-beta caryophyllene (β-caryophyllene), curcumin, honokiol, cannabinol, cannabidiol, delta 9-tetrahydrocannabinol, delta 8-tetrahydrocannabinol, hydroxy-tetrahydrocannabinol, 11-hydroxy-9-tetrahydrocannabinol, levonantradol, delta 11-tetrahydrocannabinol, tetrahydrocannabivarin, dronabinol, nabilone, ajulemic acid, triaryl bis-sulfone, luteolin, or a combination thereof.

In an embodiment, the endocannabinoids and/or the cannabinoids used in the analgesic compositions may be in the form of particles (e.g., nanoparticles and/or microparticles). In an embodiment, at least one of the endocannabinoids or the cannabinoids used in the analgesic compositions may be in the form of particles. Nanoparticles are small particles with dimensions in the nanometer range. Microparticles have particle sizes of about 100.1 to about 100,000 nanometers. These types of particles are defined further below. These particles (nanoparticles and microparticles) may have different geometries such as, for example, spherical, platelet-like, ellipsoidal, rod-like (e.g., elongated or cylindrical shape), cuboidal, triangular shape, core and shell, star-shaped particles (e.g., the particles have multiple arms radiating from a central core, or the like. In an embodiment, the particles have platelet-like shapes (i.e., they are disc shaped).

The particle size distribution of particles may be unimodal or multimodal. Multimodal particle size distributions may include binodal, trinodal or multinodal distributions. Multinodal may include 4 or more distributions in particle size.

As noted above, the analgesic composition can contain particles. These particles include nanoparticles, microparticles, or simultaneously contain both nanoparticles and microparticles. In other words, the analgesic composition can contain particles that have particle sizes that extend from the nanoparticle range to the microparticle range. In an embodiment, the nanoparticles have a particle size of less than about 100 nanometers, preferably about 2 to about 80 nanometers and more preferably about 5 to about 50 nanometers. The microparticles have a size of about 100.1 to about 100,000 nanometers, preferably about 200 to about 800 nanometers, and more preferably about 250 to about 400 nanometers.

The endocannabinoids may be present in the analgesic composition in an amount of about 0.1 to about 97 weight percent (wt %), about 0.2 to about 85 wt %, about 1 to about 80 wt %, about 2 to about 70 wt %, about 3 to about 60 wt %, about 3.5 to about 55 wt %, about 4 to about 50 wt %, about 4.5 to about 45 wt %, about 5 to about 40 wt %, about 5.5 to about 35 wt %, about 6 to about 30 wt %, about 6.5 to about 25 wt %, about 7 to about 20 wt %, about 7.5 to about 18 wt %, about 8 to about 15 wt %, and about 9 to about 14 wt %, based on a total weight of the analgesic composition. The cannabinoid may be present in the analgesic composition in an amount of 0 to about 97 weight percent (wt %), 0.1 to about 95 weight percent (wt %), about 0.2 to about 85 wt %, about 1 to about 80 wt %, about 2 to about 70 wt %, about 3 to about 60 wt %, about 3.5 to about 55 wt %, about 4 to about 50 wt %, about 4.5 to about 45 wt %, about 5 to about 40 wt %, about 5.5 to about 35 wt %, about 6 to about 30 wt %, about 6.5 to about 25 wt %, about 7 to about 20 wt %, about 7.5 to about 18 wt %, about 8 to about 15 wt %, and about 9 to about 14 wt %, based on a total weight of the analgesic composition.

In a preferred embodiment, the endocannabinoids and/or cannabinoids may be used in the analgesic composition in an amount of 10 to 80 wt %, preferably 20 to 60 wt %, and preferably 35 to 45 wt %, based on a total weight of the analgesic composition.

The analgesic composition may optionally contain a local anesthetic. The local anesthetic facilitates a reduction in pain. The local anesthetics may induce a reversible loss of sensation in a specific area of the body. They work by blocking nerve signals in the vicinity of the application, leading to temporary numbness or loss of pain sensation. In an embodiment, the local anesthetic can act synergistically with the endocannabinoid and/or the cannabinoid to provide more efficient and safer therapy. In an embodiment, the endocannabinoid and/or the cannabinoid can serve as a carrier for the sustained delivery of a local anesthetic. The endocannabinoid and/or the cannabinoid can reduce the toxicity of the local anesthetic when the analgesic composition is administered in vivo periarticularly and/or intraarticularly to a joint.

Examples of local anesthetics include lidocaine (xylocaine), bupivacaine (marcaine), mepivacaine (carbocaine), ropivacaine (naropin), procaine (novocain), articaine (septocaine), chloroprocaine, Qx-314, or the like, or a combination thereof. A preferred local anesthetic is QX-314. In an embodiment, a preferred local anesthetic for use in a joint such as the knee is bupivacaine. In an embodiment, the local anesthetic may be entirely soluble in the liquid. In another embodiment, the local anesthetic may be present as particles (nano or microparticles in the analgesic composition.

In an embodiment, the analgesic composition may contain the local anesthetic in an amount of 0 to about 75 weight percent (wt %), about 0.005 to about 50 wt %, about 0.1 to about 45 wt %, about 1 to 47 wt %, about 2 to about 40 wt %, about 3 to about 35 wt %, based on a total weight of the analgesic composition.

In an embodiment, when an endocannabinoid, a lipid and a local anesthetic are used in an analgesic composition, each of the endocannabinoid, the lipid and the local anesthetic may each be used in an amount of about 1 to about 90 wt %, preferably about 20 to about 40 wt %, preferably about 30 to about 35 wt %, based on a total weight of the endocannabinoid, the lipid and the local anesthetic.

In an embodiment, in a suspension, the endocannabinoid and/or the cannabinoid and/or the local anesthetic can be present in an amount of about 0.1 to about 25 volume percent, based on a total volume of the entire suspension.

The liquid present in the analgesic composition is preferably one that is biocompatible and does not solvate the endocannabinoid. Water or an aqueous solution is a preferred liquid for the endocannabinoid particles. Some endocannabinoids are known to be crystalline. Endocannabinoids are also water-insoluble and would have to be solvated in an organic solvent (that may not be compatible with the surrounding tissue into which it is injected). It is therefore desirable and efficacious to convert the endocannabinoids into particles in an aqueous liquid. Suitable liquids are water, phosphate-buffered saline (PBS) or saline. The saline solution preferably has a concentration of 0.9% weight per unit volume (w/v) sodium chloride (i.e., it is considered isotonic, meaning it has a similar osmotic pressure to bodily fluids). The saline solution may be hypertonic (higher concentration of sodium chloride than 0.9%) or hypotonic (lower concentration of sodium chloride than 0.9%) if desired.

Other organic solvents that are not toxic may be mixed with the liquid in small quantities if desired. Examples of such organic solvents include ethanol, polyethylene glycol, polypropylene glycol, chloroform, or a combination thereof. These organic solvents may be used to alter the size of the particles depending upon the application.

The liquid is present in the analgesic composition in an amount of about 0.5 to about 99.9 wt %, about 1 to about 95 wt %, about 5 to about 90 wt %, about 10 to about 80 wt %, about 15 to about 75 wt %, about 20 to about 70 wt %, about 25 to about 70 wt %, about 30 to about 65 wt %, about 35 to about 60 wt %, about 40 to about 55 wt %, about 45 to about 50 wt %, about 20 to about 60 wt %, and about 25 to about 50 wt %, based on a total weight of the analgesic composition.

In an embodiment, the endocannabinoid and/or the local anesthetic may each be used in an amount of about 0.01 to about 75 wt %, about 0.1 to about 1 wt %, and about 0.15 to about 1.5 wt %, based on a total weight of the analgesic composition, while the liquid may be used in an amount of about 95 to 99.9 wt %, about 96 to 99.8 wt %, based on a total weight of the analgesic composition.

### Manufacturing of the Particles

As noted above, the endocannabinoid and/or the cannabinoid may be present in the analgesic composition in the form of particles (e.g., nanoparticles or microparticles). The charged lipid and/or the supporting lipid along with/without endocannabinoid and/or the cannabinoid (and optional local anesthetic) are mixed and dispersed in a first organic solvent. In an exemplary embodiment, the first organic solvent is an alcohol. The first organic solvent is then evaporated in vacuum overnight leaving behind a powder or film.

While the first solvent is listed above as being an alcohol, other solvents such as, for example ketones, dimethylsulfoxide, dimethylformamide, tetrahydrofuran, acetone, methyl ethyl ketone, ethylene glycol, chloroform, and the like, or a combination thereof may be used. Examples of alcohol include methanol, ethanol, propanol, butanol, or a combination thereof. It is desirable to remove all traces of solvents prior to the homogenization process that is described below.

The charged lipid and/or the supporting lipid along with/without endocannabinoid and/or the cannabinoid (along with optional local anesthetic) and any supporting lipid is dissolved in a first solvent. The first solvent may be an alcohol such as methanol. The dissolution temperature is about 50 to about 80° C., preferably about 60 to about 75° C. The first solvent is evaporated under a vacuum.

The sample after the removal of methanol is mixed with sterile filtered water. For crystalline lipids, hot homogenization is performed. The hot homogenization process is conducted at a temperature of above the melting point of the lipid. For example, for PEA, a temperature of about 105 to about 110° C. was used under rotary agitation of about 1,000 to about 15,000 revolutions per minute (rpm), preferably about 2, 000 to about 10,000 rpm for a period of time of about 2 to about 30 minutes, preferably about 4 to about 15 minutes to form particles. In an embodiment, the particles in the water may constitute the analgesic composition. Alternatively, a local anesthetic may be added to the particle solution to form the analgesic solution. The local anesthetic may dissolve in the water (i.e., be soluble in the analgesic solution) or alternatively, be present in the form of particles.

### Administration of Analgesic Composition in or Around a Joint

The analgesic composition may be administered (i.e., injected) directly into a subject (a living being) at the site of pain, for instance, in a therapeutically effective amount periarticularly and/or intraarticularly around a damaged joint. In a preferred embodiment, the analgesic composition is administered in a therapeutically effective amount periarticularly to reduce inflammation, pain and/or swelling. In another embodiment, the analgesic composition without the local anesthetic may be administered (locally) in a therapeutically effective amount (dose) periarticularly and/or intraarticularly followed by a second dose that contains the local anesthetic also administered periarticularly and/or intraarticularly. In other words, the analgesic composition and the local anesthetic may be administered simultaneously (as a single therapeutically effective amount) or sequentially in two separate doses—the first dose being the analgesic composition and the second dose being the local anesthetic or vice versa.

In an embodiment, the analgesic composition may be delivered in a single dose in a therapeutically effective amount periarticularly. The single dose has been found to be as efficacious as two or more doses delivered periarticularly, preferably as efficacious as four or more doses delivered periarticularly.

In an embodiment, the analgesic composition periarticularly provides a prolonged hypoalgesic/analgesic effect for the local anesthetic.

The unique synergistic effects of the lipids in conjunction with a local anesthetic enhances the analgesic efficacy and reduces the side effects of the local anesthetic. This facilitates safer and more efficient local anesthetic formulations. The formulation can replace current treatments where a local anesthetic is used, in order to decrease its inflammatory effect and prolong its anesthetic-hypoalgesic-analgesic effect. For instance, that analgesic composition disclosed herein may be used for pain relief for the following:
a. General surgery: colon, stomach, hernia, kidney, weight loss
b. Orthopedic surgery/Sports medicine: knee, hip, spine, foot, ankle, hand, shoulder
c. Oral surgery/dental procedures: wisdom teeth, jaw
d. OB-GYN/Breast surgery: hysterectomy, fibroid, breast, C-section (Caesarean section)
e. Anesthesia/Pain medicine: nerve blocks, epidurals

The analgesic composition is advantageous in that it can be administered in the periarticular regions around the knee to significantly prolong the analgesic efficacy of the local anesthetic. This indicates a synergy between the local anesthetic and the lipids.

The analgesic compositions disclosed herein and the method of manufacture thereof are exemplified by the following non-limited examples.

### EXAMPLE

### Example 1

This example was conducted to demonstrate the manufacturing of the analgesic composition nanoparticles using two lipids—a charged lipid and a supporting lipid. An endocannabinoid is also added to the analgesic composition. In this example, endocannabinoid like lipids and/or supporting lipid such as phospholipids along with charged lipids are mixed and dispersed in an organic solvent (chloroform). The organic solvent is evaporated in a vacuum overnight. The samples were subjected to homogenization at a temperature above the melting point of the lipids to form the nanoparticle formulation.

In this example, palmitoylethanolamide [PEA] (the endocannabinoid), the supporting lipid-1,2-dipalmitoyl-sn-glycero-3-phosphocholine [DPPC] and the charged lipid-L-α-phosphatidic acid [PA] were measured in an initial mass ratio of 1:1:0.5 and were dissolved in chloroform.

The mixture was vortexed and if needed heated up to 60° C. to completely dissolve and disperse the lipids. The sample was placed in a vacuum oven for overnight removal of organic solvent. The sample was subjected to homogenization at temperature, preferably room temperature −110° C. depending on the melting point of the lipids at atmospheric pressure. Filtered water at the same temperature used was added to the sample and the sample homogenized at 10,000 rpm for 3-10 minutes to result in the nanoparticle formulation. The formulation was characterized by Transmission Electron Microscopy [TEM].

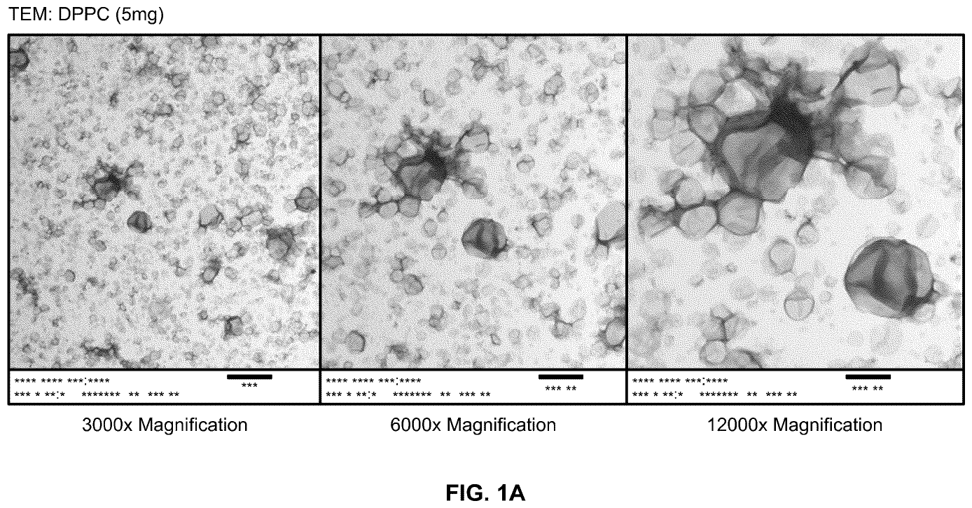

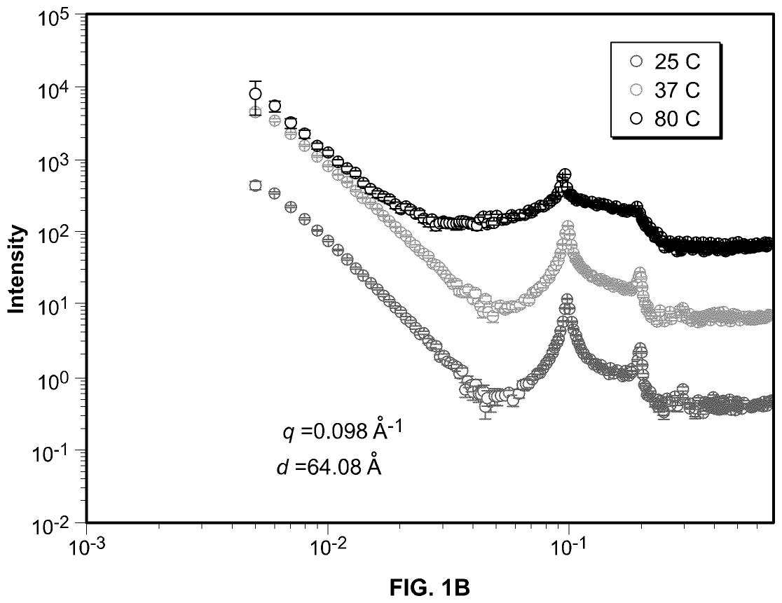

FIG. 1A shows properties of DPPC nanoparticles. The transmission electron microscopy (TEM) image indicates vesicular structures of different sizes taken at magnifications of 3000×, 6000× and 12,000×. FIG. 1B is a graph that shows small angle X-ray scattering (SAX) images showing the synchronous peaks indicating the formation of bilayers and stacking of bilayers to form multilamellar vesicles (MLVs).

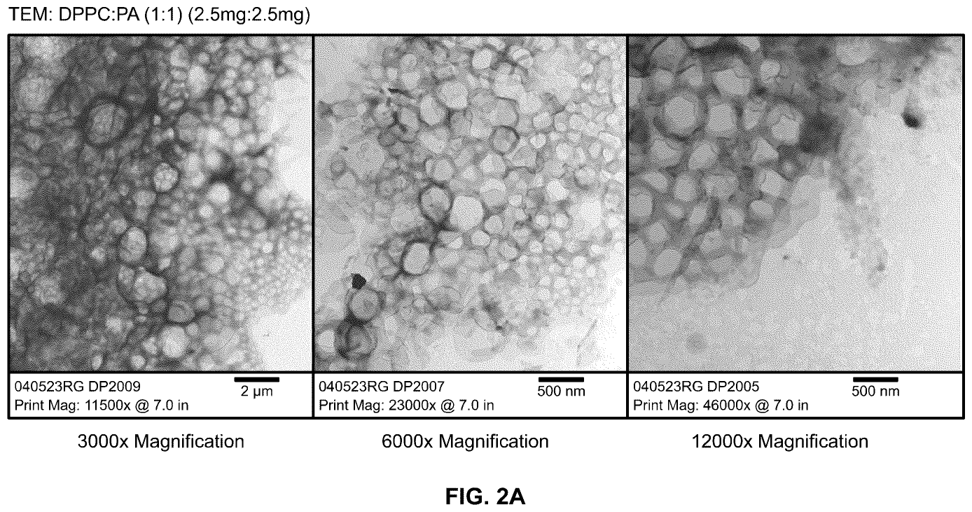

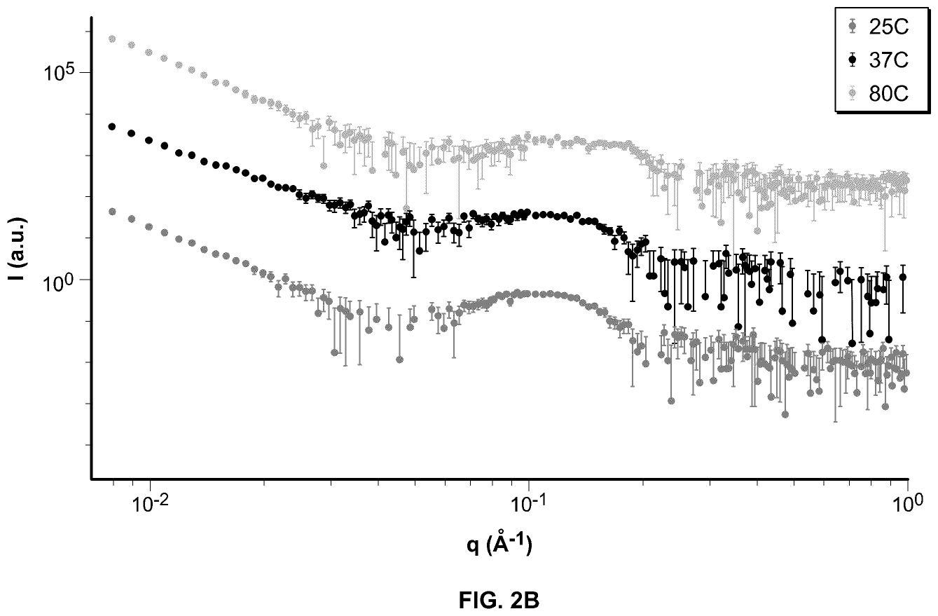

FIG. 2A shows the properties of charged particles, DPPC:PA at a mass ratio of DPPC:PA=1:1. The DPPC:PA nanoparticles shows vesicular morphology. FIG. 2B is a small angle X-ray scattering (SAX) spectrum that shows the multilamellar structure has been effectively decoupled in the presence of PA. The broad peak representing bilayer thickness becomes broadened which is different from that of DPPC bilayer, suggesting a wider distribution of bilayer thickness after the inclusion of PA. The width of the distribution increases with increased temperature.

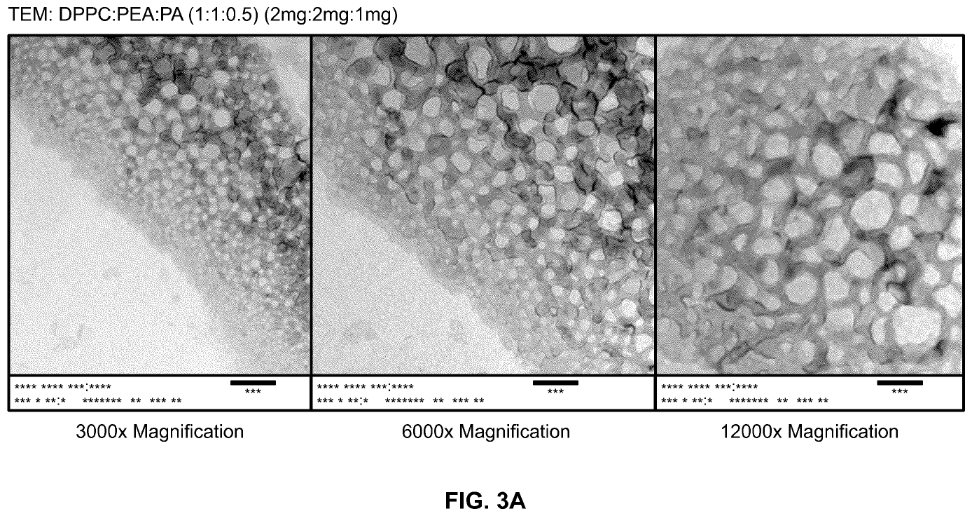

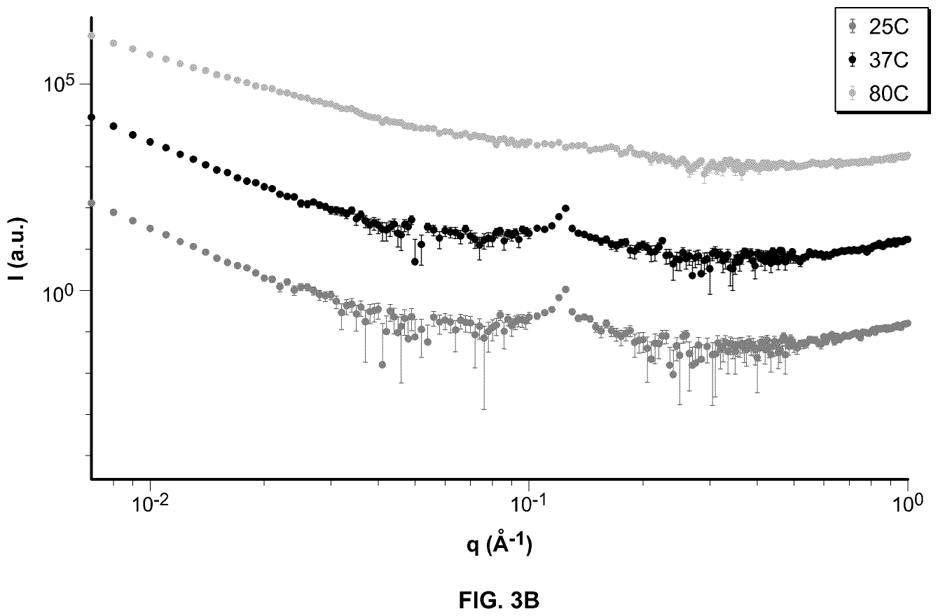

FIG. 3A shows the properties of charged DPPC-PEA-PA NPs at a mass ratio of DPPC:PEA:PA=1:1:0.5. The TEM image indicates nanoparticles with vesicular morphology. FIG. 3B is a small angle X-ray scattering spectrum, which indicates that the addition of PEA would re-induce the stacking of the bilayers (forming MLVs). The stacking is diminished upon elevation of temperature.

### Example 2

This example was conducted to demonstrate the complexation of a local anesthetic with a lipid. The local anesthetic is QX-314. The DPPC:PA (Mass ratio=1:1) nanoparticles from Example 1 were complexed with QX-314 by incubating 0.5 mg of QX-314 with 1 mL of 0.25% (w/v) lipid solution in a 37° C. room on a gentle shaker. After a period of 1 hour to 24 hours, the formulation was centrifuged at 2000 to 4000 rpm for 5 minute), and the supernatant collected.

High Performance Liquid Chromatography (HPLC) was used to assess the amount of QX-314 remaining in the supernatant after complexing with the NPs. The conditions for HPLC were as follows. The mobile phase consisted of 50 mM phosphate buffer, with 0.16% triethylamine, pH 4.0 buffer:methanol:acetonitrile=60:30:10 vol/vol/vol. The chromatographic conditions were: 30 μL of solution injected, 40° C. constant temperature, flow rate of 0.3 ml/min, and wavelength detection of 210 nm. The column used was a Supelco C18 Column 25 cm×4.6 mm.

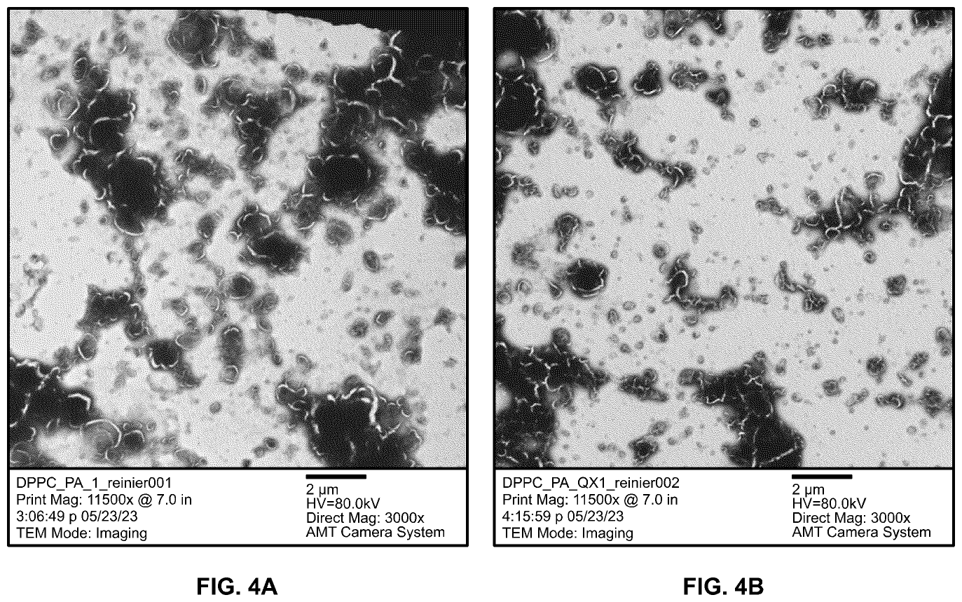

FIG. 4A shows the TEM micrograph of DPPC:PA nanoparticles before complexation while FIG. 4B is a micrograph that shows complexation with QX-314. The DPPC:PA and DPPC:PA:QX-314 show similar vesicular morphology indicating that complexation has no impact on nanoparticle morphology.

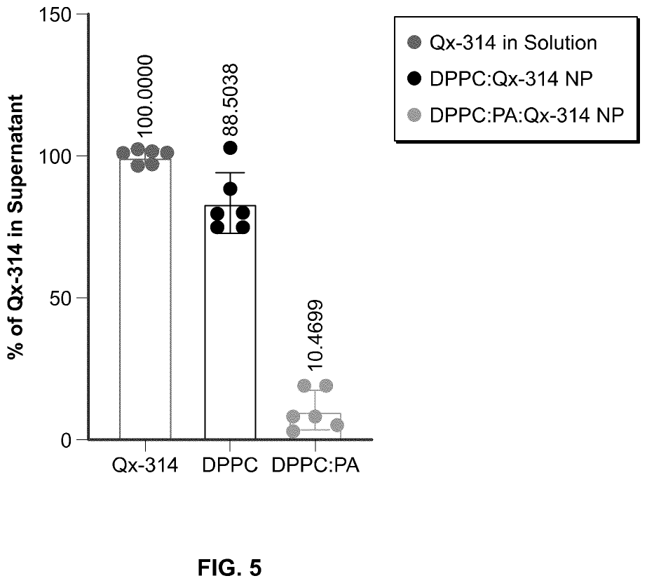

FIG. 5 shows the percentage of QX-314 in the supernatant after 1 hour of incubation followed by 3000 RPM. The graph indicates that only ~10% of the QX-314 was in the supernatant for DPPC-PA, whereas the DPPC nanoparticle showed ~89% of the QX-314 in the supernatant demonstrating the significant ability of the DPPC-PA charged nanoparticles to complex with the local anesthetic molecule. A period of approximately 1 hour or less may be used to support efficient complexation.

### Example 3

The DPPC:PA (mass ratio=1:1) nanoparticles were complexed with QX-314 by incubating 0.5 mg of QX-314 with 1 mL of 0.25% (w/v) lipid solution in a 37° C. room on a gentle shaker. After 1 hour, the formulation was transferred to a dialysis membrane and incubated in 20 ml of ultrapure water. The amount of QX-314 released into the water was measured as a function of time using HPLC as described above. Aqueous solution of QX-314 in dialysis membrane was used as the control. A representative image of positively charged molecule (QX-314) complexed with negatively charged lipid (L-α-phosphatidic acid) is shown below.

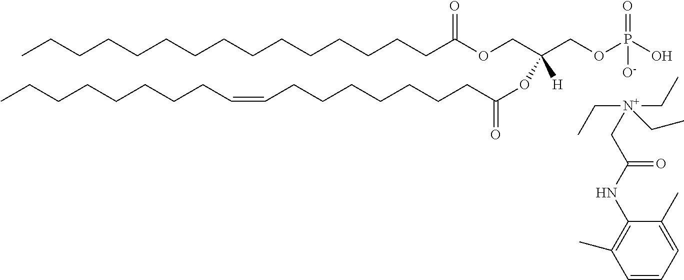 [SMILES: CCCCCCCC/C=C\CCCCCCCC(=O)O[C@H](COC(=O)CCCCCCCCCCCCCCC)COP(=O)([O-])O ; CC[N+](CC)(CC)CC(=O)Nc1c(C)cccc1C]

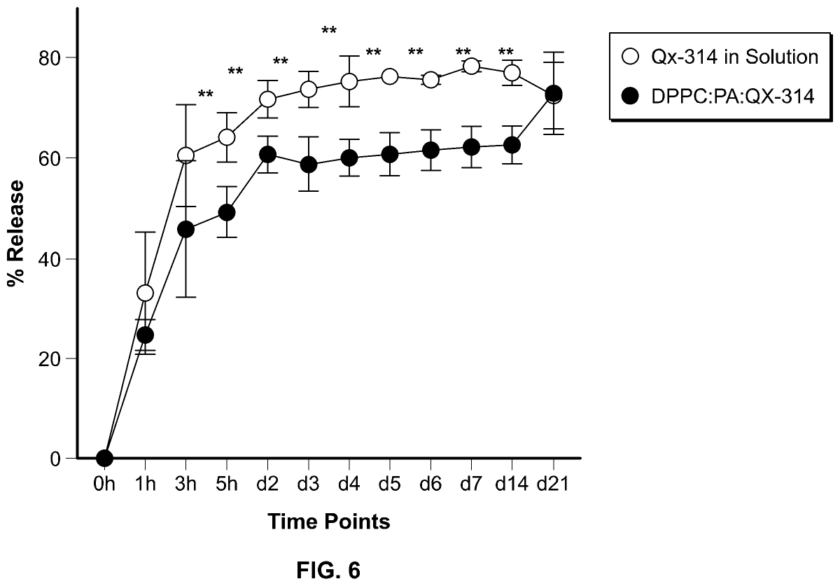

FIG. 6 is a graph that shows the percentage of QX-314 released in the water as a function of time from DPPC-PA nanoparticles and from QX-314 solution. The graph indicates that QX-314 solution showed a burst release within the first 3 hr. DPPC-PA nanoparticles on the other hand showed a sustained release with statistical significance up to ~14 days.

Unless otherwise defined, all technical and scientific terms used herein have the same meaning as commonly understood by one of ordinary skill in the art. For example, any nomenclatures used in connection with, and techniques of, cell and tissue culture, molecular biology, immunology, microbiology, genetics, and protein and nucleic acid chemistry and hybridization described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

Compounds and materials are described using standard nomenclature. Unless defined otherwise, all technical and scientific terms used herein have the same meaning as is commonly understood by one of skill in the art to which this invention belongs. The following terms are used to describe the invention of the present disclosure. In instances where a term is not specifically defined herein, that term is given an art-recognized meaning by those of ordinary skill applying that term in context to its use in describing the present disclosure.

The use of the terms “a” and “an” and “the” and similar referents (especially in the context of the following claims) are to be construed to cover both the singular and the plural, unless otherwise indicated herein or clearly contradicted by context. By way of example, “an element” means one element or more than one element.

As used herein, the term “substantially” means to a great or significant extent, but not completely.

It should also be understood that, in certain methods described herein that include more than one step or act, the order of the steps or acts of the method is not necessarily limited to the order in which the steps or acts of the method are recited unless the context indicates otherwise. Furthermore, the terms first, second, etc., as used herein are not meant to denote any particular ordering, but simply for convenience to denote a plurality of, for example, layers.

The terms “comprising”, “having”, “including”, and “containing” are to be construed as open-ended terms (i.e., meaning “including, but not limited to”) unless otherwise noted.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a nonlimiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The phrase “one or more,” as used herein, means at least one, and thus includes individual components as well as mixtures/combinations of the listed components in any combination.

The terms “about” or “approximately,” as used herein, is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within +10% or 5% of the stated value. Other than in the operating examples, or where otherwise indicated, all numbers expressing quantities of ingredients and/or reaction conditions are to be understood as being modified in all instances by the term “about,” meaning within 10% of the indicated number (e.g., “about 10%” means 9%-11% and “about 2%” means 1.8%-2.2%).

Recitation of ranges of values are merely intended to serve as a shorthand method of referring individually to each separate value falling within the range, unless otherwise indicated herein, and each separate value is incorporated into the specification as if it were individually recited herein. All ranges disclosed herein include both end points as discrete values as well as all integers and fractions specified within the range. For example, a range of 0.1-2.0 includes 0.1, 0.2, 0.3, 0.4 . . . 2.0. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed.

No language in the specification should be construed as indicating any non-claimed element as essential to the practice of the invention as used herein. All ranges and amounts given herein are intended to include subranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints. Further, it is understood that when an amount of a component is given, it is intended to signify the amount of the active material unless otherwise specifically stated.

As used herein, the term “administering” means the actual physical introduction of a composition into or onto (as appropriate) a subject, a host, or cell. Any and all methods of introducing the composition into the subject, host or cell are contemplated according to the invention; the method is not dependent on any particular means of introduction and is not to be so construed. Means of introduction are well-known to those skilled in the art, and also are exemplified herein. “Providing” means giving, administering, selling, distributing, transferring (for profit or not), manufacturing, compounding, or dispensing.

The term “subject” or “patient” is used herein to refer to an animal, such as a mammal, including a primate (such as a human, a non-human primate, e.g., a monkey, and a chimpanzee), a non-primate (such as a cow, a pig, a camel, a llama, a horse, a goat, a rabbit, a sheep, a hamster, a guinea pig, a cat, a dog, a rat, a mouse, and a whale), a bird (e.g., a duck or a goose), and a shark. In an embodiment, the subject or patient is a human subject or a human patient, such as a human being treated or assessed for a disease, disorder or condition, a human at risk for a disease, disorder or condition, a human having a disease, disorder or condition, and/or human being treated for a disease, disorder or condition as described herein. In one embodiment, the subject is about 1, 2, 3, 4, 5, 6, 7, 8, 9, or 10 years of age. In another embodiment, the subject is about 5-10, 10-15, 15-20, 20-25, 25-30, 30-35, 35-40, 40-45, 45-50, 50-55, 55-60, 60-65, 65-70, 70-75, 75-80, 80-85, 85-90, 90-95, 95-100 years of age. Values and ranges intermediate to the above recited ranges are also intended to be part of this invention. In addition, ranges of values using a combination of any of the above-recited values as upper and/or lower limits are intended to be included. As used herein, a subject is “in need of treatment” if such subject would benefit biologically, medically, or in quality of life from such treatment. A subject in need of treatment does not necessarily present symptoms, particular in the case of preventative or prophylaxis treatments.

While the invention has been described with reference to some embodiments, it will be understood by those skilled in the art that various changes may be made and equivalents may be substituted for elements thereof without departing from the scope of the invention. In addition, many modifications may be made to adapt a particular situation or material to the teachings of the invention without departing from essential scope thereof. Therefore, it is intended that the invention not be limited to the particular embodiments disclosed as the best mode contemplated for carrying out this invention, but that the invention will include all embodiments falling within the scope of the appended claims.

1. An analgesic composition comprising:
a nanoparticle and/or a microparticle that comprises a charged lipid and/or a supporting lipid; where the charged lipid comprises an electrically charged group.

2. The analgesic composition of claim 1, where the charged lipid is L-α-phosphatidic acid [PA], oleoyl-L-α-lysophosphatidic acid, 1,2-diarachidonoyl-sn-glycero-3-[phosphor-rac-(1-glycerol)] (20:4 PG); tetradecanoic acid, 1,1′,1″,1″-{(2R,14R)-5,8,11,-trihydroxy-5,11-dioxido-4,6,10,12-tetraoxa-5,11-diphosphapentadecane-1,2,14,15-tetrayl] ester (Tetramyristoylcardiolipin); (R)-2,3-bis(((9Z,12Z)-octadeca-9,12-dienoyl)oxy)propyl ((1S,2R,3R,4S,5S,6R)-2,3,4,5,6-pentahydroxycyclohexyl) phosphate (Phosphatidylinositol); (1,2-dioleoyl-3-trimethylammonium-propane (POPS); N-(2-hydroxyethyl)-N,N-dimethyl-2,3-bis(oleoyloxy) propan-1-aminium bromide (DORI); O,O′-ditetradecanoyl-N-(α-trimethylammonioacetyl) diethanolamine chloride (DC-6-14); 1,2-di-O-octadecenyl-3-trimethylammonium propane (DOTMA); N1-[2-((1S)-1-[(3-aminopropyl)amino-4-[di(3-amino-propyl)amino]butylcarboxamido)ethyl]-3,4-di[oleyloxy]-benzamide (MVL5), or a combination thereof.

3. The analgesic composition of claim 1, where the charged lipid is L-α-phosphatidic acid.

4. The analgesic composition of claim 1, where the supporting lipid comprises 1,2-dipalmitoyl-sn-glycero-3-phosphocholine [DPPC], 1,2-dimyristoyl-sn-glycero-3-phosphocholine [DMPC], 1,2-distearoyl-sn-glycero-3-phosphocholine [DSPC], 1,2-dioleoyl-sn-glycero-3-phosphocholine [DOPC], or a combination thereof.

5. The analgesic composition of claim 1, further comprising an endocannabinoid and/or a cannabinoid.

6. The analgesic composition of claim 5, where the endocannabinoid comprises at least one of anandamide (AEA) 2-arachidonoylglycerol (2-AG), 2-oleoyl glycerol (2-OG), palmitoylethanolamide (PEA), oleoylethanolamide (OEA), linoleoylethanolamide (LEA), adelmidrol (N,N′-Bis(2-hydroxyethyl) nonanediamide), N-stearoyl ethanolamine (SEA), N-arachidonoyl glycine (NArGly), N-arachidonoylserine (NArS), N-acyl taurine, N-acyl dopamine, N-acyl alanine, or a combination thereof.

7. The analgesic composition of claim 5, where the cannabinoid comprises at least one of cannabigerol (CBG), cannabichromene (CBC), trans-beta caryophyllene (β-caryophyllene), curcumin, honokiol, cannabinol, cannabidiol, delta 9-tetrahydrocannabinol, delta 8-tetrahydrocannabinol, hydroxy-tetrahydrocannabinol, 11-hydroxy-9-tetrahydrocannabinol, levonantradol, delta 11-tetrahydrocannabinol, tetrahydrocannabivarin, dronabinol, nabilone, ajulemic acid, triaryl bis-sulfone, luteolin, or a combination thereof.

8. The analgesic composition of claim 1, where the charged lipid is present in an amount of in an amount of 10 to 40 weight percent, based on a total weight of the analgesic composition.

9. The analgesic composition of claim 1, where the supporting lipid is present in an amount of in an amount of 10 to 80 weight percent, based on a total weight of the analgesic composition.

10. The analgesic composition of claim 1, where the endocannabinoid and/or the cannabinoid is present in an amount of 10 to 80 weight percent, based on a total weight of the analgesic composition.

11. The analgesic composition of claim 1, further comprising a local anesthetic in an amount of up to 75 wt %, based on a total weight of the analgesic composition.

12. The analgesic composition of claim 11, where the local anesthetic is complexed with the charged lipid.

13. The analgesic composition of claim 11, where the nanoparticles are vesicular structures that comprise bilayers.

14. The analgesic composition of claim 13, where the nanoparticles comprise multilamellar vesicles, where the multilamellar vesicles comprise stacked bilayers.

15. A method of manufacturing particles, the method comprising:
dissolving a lipid in a first solvent; where the lipid is a charged lipid, a supporting lipid, or a combination of the charged lipid and the supporting lipid
evaporating the first solvent to leave behind a powder or film;
adding to the powder or film a second solvent;
heating the second solvent containing the powder and the film to a temperature greater than about 100° C. or to a temperature higher than the melting point of the lipid; and
agitating the aqueous solution with a rotary agitator at about 1,000 to about 15,000 revolutions per minute to form the particles; where the particles are nanoparticles and/or microparticles.

16. The method of claim 15, where the first solvent is chloroform and where the second solvent is water.

17. A method of reducing joint inflammation, swelling and/or pain in a subject, the method comprising:
administering a therapeutically effective amount an analgesic composition to the subject; where the analgesic composition comprises a nanoparticle and/or a microparticle that comprises a charged lipid and/or a supporting lipid; where the charged lipid comprises an electrically charged group.

18. The method of claim 17, where the administering is conducted periarticularly.

19. The method of claim 17, where the administering is conducted intraarticularly.

20. The method of claim 17, where the analgesic composition further comprises one or more of an endocannabinoid, a cannabinoid or a local anesthetic.
